(19) United States
(12) Patent Application Publication
(10) Pub. No.: US 2024/0082457 A1
(43) Pub. Date: Mar. 14, 2024

(54) HARD TISSUE REPAIR ADHESIVE COMPOSITION AND HARD TISSUE REPAIR KIT THEREOF

(71) Applicant: JIANGXING JINGYIN BIOTECHNOLOGY CO., LTD, Jiaxing (CN)

(72) Inventors: Weiping ZENG, Beijing (CN); Xianghao ZHANG, Beijing (CN); Tuanfeng ZHOU, Beijing (CN); Yang WANG, Beijing (CN); Chenggang DUAN, Beijing (CN)

(21) Appl. No.: 18/259,967

(22) PCT Filed: Mar. 29, 2021
(86) PCT No.: PCT/CN2021/083557

§ 371 Date: Jun. 29, 2023

(30) Foreign Application Priority Data
Jan. 9, 2021 (CN) 202110018795.9

(51) Int. Cl. A61L 24/06; C08F 220/14

(52) U.S. Cl. CPC A61L 24/06; C08F 220/14

(57) ABSTRACT

A hard tissue repair adhesive composition and a hard tissue repair kit comprising same. The adhesive composition comprises a polymerizable monomer (A), polymer powder (B), a polymerization initiator (C), and a filler (D). The polymerization initiator (C) comprises 100 parts by weight of alkoxydialkylboron having a purity of 97% or more and 0.2-5 parts by weight of an alcohol having a boiling point of 60° C. to 180° C. The polymerization initiator (C) does not generate smoke or coking or present ignition quality even when in contact with paper, porous fibers or the like in air, is easy to be taken in a small amount, and can provide a polymerizable composition with high polymerization activity, thereby providing an adhesive composition suitable for hard tissue repair. (A), (B), (C), (D) and all other components comprised according to requirements of the adhesive composition for hard tissue repair can be separately or mixedly stored in three or more members to form a repair kit.

### TECHNICAL FIELD

The present invention relates to an adhesive composition for hard tissue repair and kit thereof for hard tissue repair. More particularly, the present invention relates to an adhesive composition for hard tissue repair having high curability and adhesion properties and, to a polymerization initiator composition for the adhesive, which has improved safety against ignition when adhered to paper or the like.

### TECHNICAL BACKGROUND

It is well known that the adhesion composition with trialkylboron as a polymerization initiator exhibits good adhesion to body hard tissues (see patent literature 1). Trialkylboron is an extremely unstable substance in air, and reacts vigorously with oxygen to ignite spontaneously when exposed to air, and thus not suitable for general clinical use. Therefore, various techniques have been developed for improving the safety of trialkylboron.

Patent literature 2 discloses a method for improving the safety against easy ignition by adding hydrophobic and viscous substances such as vaseline, paraffin wax and organosilicon, i.e., silicone oil, to trialkylboron or derivatives thereof, adding adsorbents such as silicic acid and alumina, if desired, and making into paste.

Patent literature 3 discloses a dental or surgical adhesive using partially oxidized trialkylboron as a polymerization initiator, a product obtained by reacting trialkylboron with 0.3 to 0.9 moles of oxygen. Although, the invention of patent literature 3 proposes a polymerization initiator that minimizes the activity of trialkylboron to improve the safety against ignition, it is difficult to obtain a reactant of a certain composition since this polymerization initiator results from a liquid-gas reaction of trialkylboron with oxygen, thereby leading to the instability of the curing rate of the adhesive using it as the initiator and inhibiting ignition incompletely.

Patent literature 4 discloses a polymerization initiator obtained in a homogeneous mixture by adding organic oligomers or organic polymers such as silicone oil, wax, oligoester, oligoamide, and the like to an organoboron compound. The addition of large amounts of additives tends to cause a decrease in polymerization initiation activity, and an increase in viscosity affects the correct measuring.

Patent literature 5, 6 propose a method for improving the safety against ignition by adding polymer powder of alkyl (meth)acrylate to tributylboron or partially oxidized tributylboron to prepare a paste initiator. The addition of large amounts of additives tends to cause a reduction in polymerization initiation activity, and the paste initiator is difficult to correct measuring.

With respect to the ignition of trialkylboron, Patent literature 7 proposes a mixture based on oxyalkylalkylboron obtained by reacting trialkyl boron with an alkyl alcohol, or further adding a polar organic compound or an inert diluent material to the mixture to improve the safety of the polymerization initiator, but it is not sufficient.

Patent literature 8 proposes a method for improving the safety by adding an aprotic solvent or further adding an inert liquid or a solid organic oligomer or polymer to tributylboron or partially oxidized tributylboron. However, the addition of large amounts of additives tends to result in reduced activity of the polymerization initiator and sometimes leads to an increase in the viscosity of the initiator composition due to the addition of the organic oligomer or polymer, which causes difficulty in accurately measuring the amount.

Patent literature 9 proposes the addition of specific amounts of alkanes and alcohols to tributylboron or partially oxidized tributylboron to inhibit ignition and improve the safety. However, the effect of paraffins remaining in the adhesive that cannot be absorbed and decomposed by the human body on the health of the human body is unknown when the initiator is used, and the addition of large amounts of additives results in a reduction in the activity of the polymerization initiator.

### PATENT LITERATURE LIST

Patent document 1: JPS42-14318
Patent document 2: JPS48-11892
Patent document 3: JPS49-5143
Patent document 4: JPS58-084803
Patent document 5: JPH3-264509
Patent document 6: CN1642933A
Patent document 7: JPH5-253284
Patent document 8: JPH9-110913
Patent document 9: CN1909870A

To sum up, there is still a room for the improvement in the initiator or initiator composition of the adhesive composition for hard tissue repair of the prior art in terms of use safety, use convenience, and biosafety.

### SUMMARY OF THE INVENTION

It is an object of the present invention to provide a polymerization initiator composition which does not cause smoking, coking or exhibits ignition even when contacting paper, porous fibers or the like in the air, has high flowability, can be easily and accurately measured in a small amount, reduces adverse effects on the human body, and can impart high polymerization activity to the polymerizable composition so as to cure the polymerizable composition in a short time, thereby an adhesive composition suitable for hard tissue repair is provided.

In order to achieve the above object, an organoboron compound with a high purity and a specific structure and a small amount of specific alcohol are used to inhibit ignition and maintain its high flowability and high polymerization activity, and thus, the present invention was accomplished. The technical embodiments adopted by the invention are as follows:

An adhesive composition for hard tissue repair, characterized in that it comprises:
a polymerizable monomer (A), a polymer powder (B), and a polymerization initiator (C), wherein the polymerization initiator (C) comprises 100 parts by weight of alkoxydialkyl boron having a purity of 97% or more, 0.2-5 parts by weight of an alcohol having a boiling point of 60 to 180° C.; wherein the polymerizable monomer (A) is a (meth)acrylate ester or a combination of a (meth)acrylate ester and a polymerizable monomer containing an acidic group; wherein the polymer powder (B) is at least one polymer selected from homopolymer of alkyl (meth)acrylate, copolymer between alkyl (meth)acrylates, copolymer of alkyl (meth)acrylate with other polymerizable monomer, copolymer of alkyl (meth)acrylate with alkylene di(meth)acrylate, and copolymer of alkyl (meth)acrylate with diene monomer;
wherein the alkoxydialkylboron comprised in the polymerization initiator (C) is butoxydibutylboron and the alcohol comprised in the polymerization initiator (C) is ethanol;
the adhesive composition for hard tissue repair comprises 20-70 parts by weight of the polymerizable monomer (A), 20-70 parts by weight of the polymer powder (B), and 1-20 parts by weight of the polymerization initiator (C), wherein the total amount of the polymerizable monomer (A), the polymer powder (B), and the polymerization initiator (C) is 100 parts by weight;
wherein the composition for hard tissue repair further comprises 20-150 parts by weight of a filler (D) based on 100 parts by weight of the total amount of the polymerizable monomer (A), the polymer powder (B), and the polymerization initiator (C). The filler (D) is an inorganic filler, an organic filler, or an organic-inorganic composite filler, which is insoluble or non-swellable in the polymerizable monomer (A);
A kit for hard tissue repair comprising the adhesive composition for hard tissue repair of the present invention, the polymerizable monomer (A) and part of the polymer powder (B) comprised in the composition for hard tissue repair are placed in the same member, the polymerization initiator (C) is placed in one member, the remaining part of the polymer powder (B) is placed in one member, and the three members above form the kit for repair; or the polymerizable monomer (A) and the filler (D) are placed in one member, the polymer powder (B) is placed in one member, the polymerization initiator (C) is placed in one member, and the three members above form the kit for repair; or the polymerizable monomer (A) is placed in one member, the polymerization initiator (C) is placed in one mernber, the polymer powder (B) and the filler (D) are placed in one member, and three members above form the kit for repair.

The present invention can provide a polymerization initiator which does not cause smoking, coking or exhibits ignition even when contacting paper, porous fibers or the like in the air, has high flowability, can be easily and accurately measured in a small amount, reduces adverse effects on the human body, and can impart high polymerization activity to the polymerizable composition so that the polymerizable composition is cured within a short time, thereby an adhesive suitable for hard tissue repair is provided.

Based on the common knowledge, reaction activity of alkylboron with oxygen, i.e., ignitability is believed to be proportional to its polymerization initiation property. However, surprisingly, the polymerization initiator of the present invention comprising alkoxydialkyl boron of high purity and a small amount of specific alcohol having lower ignition than trialkylboron and partially oxidized trialkylboron has equivalently high polymerization initiation property. The reason for this is not clear, but as one of the reasons, it is speculated that the reaction between alkoxydialkylboron of high purity of the present invention and oxygen is relatively mild compared to trialkylboron and partially oxidized trialkylboron, and the proportion of the primary free radicals generated that are consumed by the mutual reaction without being used for the polymerization initiation is relatively small, so the generated free radicals can be effectively used for polymerization initiation.

As shown in example 1, example 2 and comparative example 1 and comparative example 2 in table 1, when the partially oxidized tributylboron and the mixture of butoxydibutylboron are contacted with the filter paper, the filter paper can be coked and even ignited, while the polymerization initiator of the present invention comprising the high-purity butoxydibutylboron and a small amount of ethanol has no smoking phenomenon and also does not cause coking or ignition of the filter paper when contacting the filter paper, at the same time, has polymerization activity equivalent to those of partially oxidized tributylboron and the mixture of butoxydibutylboron.

### DETAILED DESCRIPTION OF THE PRESENT INVENTION

### Polymerizable Monomer (A)

The polymerizable monomer (A) used in the present invention is not particularly limited as long as it can be polymerized by the polymerization initiator (C) described hereinafter. With respect to the polymerizable monomer (A), both monofunctional monomers and polyfunctional monomers may be used depending on the purpose of use.

As the polymerizable monomer (A), (meth)acrylate ester monomers and other vinyl compounds may be used for example. Considering less irritation to human body, (meth)acrylate ester monomers are preferred. In the present invention, “(meth)acrylate ester” is the generic name of acrylate and methacrylate. In addition, the monomer having an acidic group is generally excellent in adhesion to hard tissue and metal material such as titanium for hard tissue repair, and therefore, the polymerizable monomer (A) can also contain an appropriate amount of the monomer having an acidic group to improve the adhesion to hard tissue and repairing material.

As specific examples of a monofunctional (meth)acrylate ester monomer without an acidic group, alkyl (meth)acrylate such as methyl (meth)acrylate, ethyl (meth)acrylate, propyl (meth)acrylate, butyl (meth)acrylate, hexyl (meth)acrylate, 2-ethylhexyl (meth)acrylate, dodecyl (meth)acrylate, lauryl (meth)acrylate, cyclohexyl (meth)acrylate, benzyl (meth)acrylate, isobornyl (meth)acrylate; hydroxyalkyl (meth)acrylate such as 2-hydroxyethyl (meth)acrylate, 2-hydroxypropyl (meth)acrylate, 3-hydroxypropyl (meth)acrylate, 4-hydroxybutyl (meth)acrylate, 5-hydroxyamyl (meth)acrylate, 6-hydroxyhexyl (meth)acrylate, 1,2-dihydroxypropyl mono(meth)acrylate, 1,3-dihydroxypropyl mono(meth)acrylate, erythritol mono(meth)acrylate; polyethylene glycol mono(meth)acrylate such as diethylene glycol mono(meth)acrylate, triethylene glycol mono(meth)acrylate, polyethylene glycol mono(meth)acrylate, and polydipropylene glycol mono(meth)acrylate; (poly) glycol monoalkyl ether (meth)acrylate such as ethylene glycol monomethyl ether (meth)acrylate, ethylene glycol monoethyl ether (meth)acrylate, diethylene glycol monomethyl ether (meth)acrylate, triethylene glycol monomethyl ether (meth)acrylate, polyethylene glycol monomethyl ether (meth)acrylate, polypropylene glycol monoalkyl ether (meth)acrylate; fluoroalkyl (meth)acrylate such as perfluorooctyl (meth)acrylate, hexafluorobutyl (meth)acrylate; silane compounds having (meth)acryloyloxyalkyl such as gamma-(meth)acryloyloxypropyltrimethoxysilane, gamma-(meth)acryloyloxypropyltris(trimethylsiloxy) silane, and the like; (meth)acrylate having heterocycle such as tetrafurfuryl (meth)acrylate can be mentioned.

As specific examples of a polyfunctional (meth)acrylate ester monomer without an acidic group, poly(meth)acrylate of alkane polyol such as ethylene glycol di(meth)acrylate, propylene glycol di(meth)acrylate, butanediol di(meth)acrylate, neopentyl glycol di(meth)acrylate, hexanediol di(meth)acrylate, dihydroxypropyl di(meth)acrylate, trimethylolpropane tri(meth)acrylate, pentaerythritol tetra(meth)acrylate; polyoxyalkane polyol poly(meth)acrylates such as diethylene di(meth)acrylate, triethylene glycol di(meth)acrylate, polyethylene glycol di(meth)acrylate, dipropylene glycol di(meth)acrylate, polypropylene glycol di(meth)acrylate, dibutylene glycol di(meth)acrylate, dipentaerythritol hexa(meth)acrylate can be mentioned;

Alicyclic or aromatic di(meth)acrylate represented by the following general formula (1)

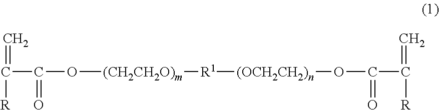

wherein R is hydrogen atom or methyl, m and n are the same or different and are numbers from 0 to 10, wherein R¹ is

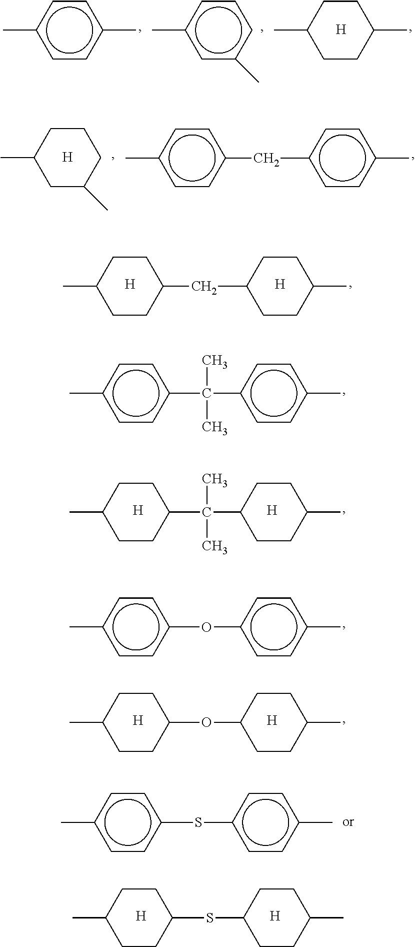

and an aliphatic or aromatic di(meth)acrylate having a hydroxyl group in the molecule represented by the following general formula (2);

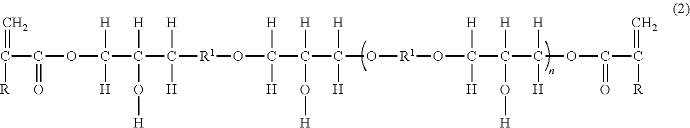

wherein R is hydrogen atom or methyl, m is a number from 0 to 10, R¹ is

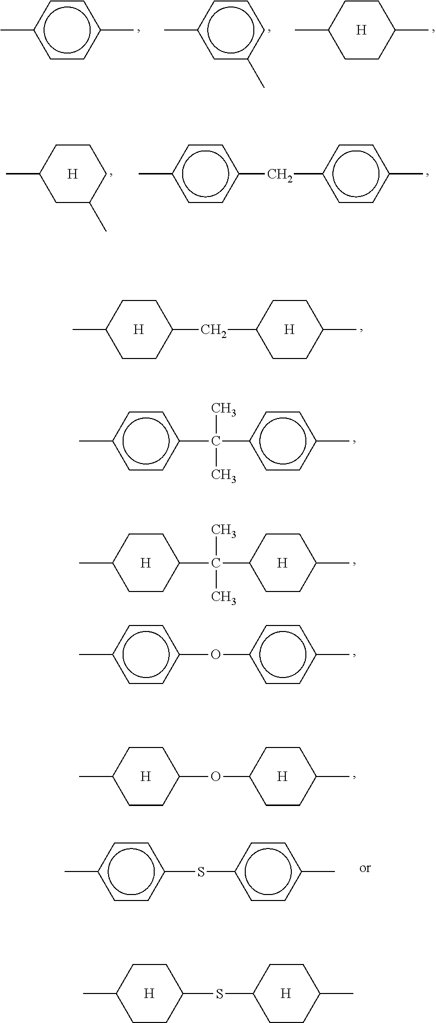

and a polyfunctional (meth)acrylate or the like having a urethane bond in the molecule represented by the following general formula (3);

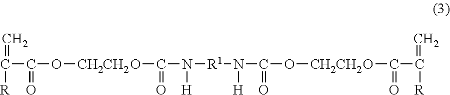

wherein R is hydrogen atom or methyl, R¹ is

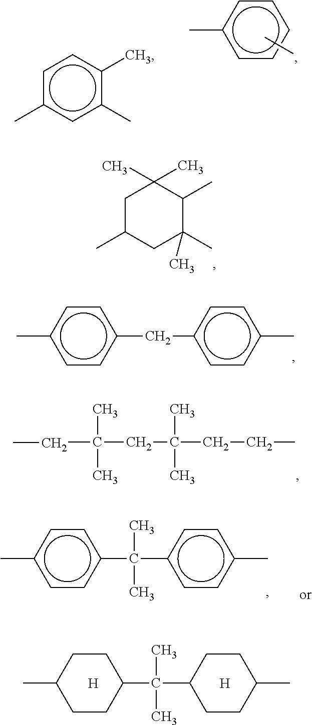

wherein, as the monofunctional (meth)acrylate, it is particularly preferred to use
alkyl (meth)acrylate such as methyl (meth)acrylate and ethyl (meth)acrylate;
hydroxyl-containing (meth)acrylate such as 2-hydroxyethyl (meth)acrylate, 1,3-dihydroxypropyl mono(meth)acrylate, erythritol mono(meth)acrylate; (meth)acrylate having ethylene glycol chain in the molecule such as triethylene glycol monomethyl ether (meth)acrylate, triethylene glycol mono(meth)acrylate, etc.

Furthermore, as the polyfunctional (meth)acrylate, di(meth)acrylate having an ethylene glycol chain in the molecule, such as triethylene glycol di(meth)acrylate, polyethylene glycol di(meth)acrylate, or the dihydroxypropyl di(meth)acrylate is particularly preferred;

Compounds represented by the following formula (1)-a:

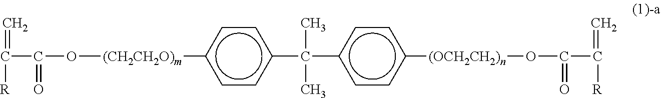

wherein R, m and n have the same definitions as in formula (1)

Compounds represented by the following formula (2)-a:

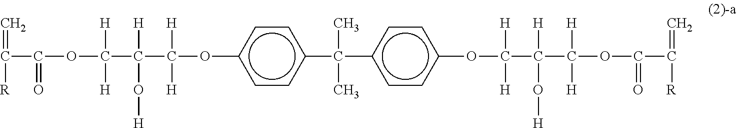

wherein R has the same definition as in formula (2);

Compounds represented by the following formula (3)-a:

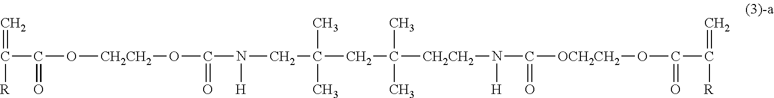

wherein R has the same definition as in formula (3). These can be used alone or in combination of two or more.

Specific examples of monomers having an acidic group are as follows:

The monomer having a carboxylic acid group or anhydride group such as (meth)acrylic acid and its anhydride, 1,4-di(meth)acryloyloxy ethyl pyromellitic acid, 6-(meth)acryloyloxyethyl naphthalene 1,2,6-tricarboxylic acid, N-(meth)acryloyl p-aminobenzoic acid, N-(meth)acryloyl anthranilic acid, N-(meth)acryloyl m-aminobenzoic acid, N-(meth)acryloyl-5-aminosalicylic acid, N-(meth)acryloyl-4-aminosalicylic acid, 4-(meth)acryloyloxyethyl trimellitic acid and its anhydride, 4-(meth)acryloyloxybutyl trimellitic acid and its anhydride, 4-(meth)acryloyloxyhexyl trimellitic acid and its anhydride, 4-(meth)acryloyloxydecyl trimellitic acid and its anhydride, 2-(meth)acryloyloxybenzoic acid, 3-(meth)acryloyloxybenzoic acid, 4-(meth)acryoyloxybenzoic acid, beta-(meth)acryloyloxyethyl succinate, beta-(meth)acryloyloxyethyl maleate, beta-(meth)acryloyloxyethyl phthalate, 11-(meth)acryloyloxy-1,1-undecanedicarboxylic acid and p-vinylbenzoic acid; the monomer having an phosphoric acid group such as (2-(meth)acryloyloxyethyl) phosphoric acid, (2-(meth)acryloyloxyethylphenyl) phosphoric acid, 10-(meth)acryloyloxydecyl phosphoric acid, and the like; the monomer having an sulfonic acid group such as p-styrenesulfonic acid, 2-acrylamido-2-methylpropanesulfonic acid and the like can be mentioned, preferably 4-methacryloyloxyethyl trimellitic acid and its anhydride. These acidic monomers can be used alone or in combination.

In the polymerizable monomer (A), preferably 80% by weight or more is a monofunctional (meth)acrylate ester monomer, more preferably 90% by weight or more is a monofunctional (meth)acrylate ester monomer, and more preferably 95% by weight or more is a monofunctional (meth)acrylate ester monomer. If the monomer (A) comprises the monomer comprising an acidic group, the amount of the monomer comprising an acidic group is preferably 1 to 20% by weight, based on 100% by weight of the total monomer (A).

The polymerizable monomer (A) is present in an amount of preferably 20 to 70 parts by weight, more preferably 30 to 65 parts by weight, and more preferably 35 to 60 parts by weight based on 100 parts by weight of the total amount of the polymerizable monomer (A), the polymer powder (B) and the polymerization initiator (C). The lower limits of the above ranges are meaningful in terms of operability, penetration of the composition into the hard tissue etc. The upper limits are meaningful in terms of curing rate, early adhesion strength, mechanical properties, etc.

### Polymer Powder (B)

The type of polymer powder (B) used in the present invention is not particularly limited, the polymer powder which is soluble or swellable in the polymerizable monomer (A) of the present invention can be used. As the polymer powder (B), (meth)acrylate ester polymer and other vinyl polymer can be used, wherein (meth)acrylate ester polymer is preferred. For example, homopolymer of alkyl (meth)acrylate, copolymer of between alkyl (meth)acrylates, copolymer of alkyl (meth)acrylate with other polymerizable monomer, copolymer of alkyl (meth)acrylate with alkylene di(meth)acrylate, and copolymer of alkyl (meth)acrylate with diene monomer can be mentioned. These can be used alone or in combination of two or more.

Examples of (meth)acrylate ester polymers can include non-crosslinked polymer such as poly methyl (meth)acrylate, poly ethyl (meth)acrylate, copolymer of methyl (meth)acrylate and ethyl (meth)acrylate, copolymer of methyl (meth)acrylate and butyl (meth)acrylate, copolymer of methyl (meth)acrylate and styrene; crosslinked polymer such as copolymer of methyl (meth)acrylate and ethylene glycol di(meth)acrylate, copolymer of methyl (meth)acrylate and triethylene glycol di(meth)acrylate, copolymer of methyl (meth)acrylate and butadiene monomer.

The polymer powder (B) is present in an amount of preferably 20 to 70 parts by weight, more preferably 30 to 65 parts by weight, and more preferably 35 to 60 parts by weight, based on 100 parts by weight of the total amount of the polymerizable monomer (A), the polymer powder (B) and the polymerization initiator (C).

### Polymerization Initiator (C)

The polymerization initiator (C) comprises 100 parts by weight of alkoxydialkylboron having a purity of 97% or more, 0.2-5 parts by weight of an alcohol having a boiling point of 60 to 180° C.

As specific example of an alkoxydialkylboron, examples can include ethoxydiethylboron, ethoxydipropylboron, ethoxydibutylboron, ethoxydiamylboron, propoxydiethylboron, propoxydipropylboron, propoxydibutylboron, propoxydiamylboron, butoxydiethylboron, butoxydipropylboron, butoxydibutylboron, preferably ethoxydibutylboron, propoxydibutylboron, butoxydibutylboron, more preferably propoxydibutylboron, butoxydibutylboron, especially butoxydibutylboron. These can be used alone or in combination of two or more.

The purity of alkoxydialkylboron used in the present invention is preferably 97% or more, more preferably 98% or more, and particularly preferably 98.5% or more. Surprisingly, when the purity of the alkoxydialkylboron used in the present invention is above a certain degree and a small amount of a special alcohol, its smoking and ignitability are significantly inhibited, but still retains the equivalent polymerization activity.

For the preparation of alkoxydialkylboron, it can be obtained by reacting trialkylboron with corresponding alcohol. Since the reaction must be carried out under an oxygen-insulated condition and is relatively sensitive to reaction temperature, generally a mixture (primary product) containing target alkoxydialkylboron as main component is obtained. The high-purity alkoxydialkylboron of the present invention is obtained by reduced pressure distillation fractionation of said primary product.

For the preparation of alkoxydialkylboron, it can also be obtained by oxidation reaction of trialkylboron with an oxidant such as oxygen. Since the reaction of trialkylboron with oxygen is liquid-gas heterogeneous reaction, reaction conditions such as gas feeding rate and stirring rate must be strictly controlled. Reaction of trialkylboron with oxygen typically results in a mixture containing the target alkoxydialkylboron as the main component (primary product). High purity alkoxydialkylboron of the present invention is obtained by reduced pressure distillation fractionation of the primary product.

As specific example of an alcohol having a boiling point of 60 to 180° C., example can include, methanol, ethanol, n-propanol or isomers thereof, n-butanol or isomers thereof, n-amyl alcohol or isomers thereof, n-hexanol or isomers thereof, and the like, more preferably an alcohol having 4 or less carbon atoms, wherein ethanol, n-propanol are particularly preferred. These alcohols can be used alone or in combination of two or more.

Based on 100 parts by weight of high-purity alkoxydialkylboron, the amount of the alcohol comprised in the polymerization initiator of the present invention must be 0.2-5 parts by weight, more preferably 0.3-4.5 parts by weight, and most preferably 0.5-4 parts by weight. Based on 100 parts by weight of high-purity alkoxydialkylboron, when the amount of the alcohol is less than 0.2 parts by weight, sufficient smoking and ignition inhibition effects cannot be obtained. When the amount of the alcohol exceeds 5 parts by weight, there is a tendency to reduce the polymerization ability of the polymerization initiator composition.

Preferably, the polymerization initiator (C) is present in an amount of 1 to 20 parts by weight, more preferably 2 to 15 parts by weight, and more preferably 3 to 10 parts by weight, based on 100 parts by weight of the total amount of the polymerizable monomer (A), the polymer powder (B), and the polymerization initiator (C).

### Filler (D)

The type of filler (D) used in the present invention is not particularly limited and inorganic fillers, organic fillers and organic-inorganic composite fillers all can be used as long as they are insoluble or non-swellable in the polymerizable monomer (A) of the present invention.

Examples of the inorganic filler useful as the filler (D) of the present invention include metal oxide powders such as zirconia, bismuth oxide, titanic, zinc oxide, and alumina particles; metal salt powders such as calcium carbonate, calcium bicarbonate, sodium carbonate, sodium bicarbonate, bismuth carbonate, calcium phosphate, zirconium phosphate, and barium sulfate; hydroxyapatite, carbonate apatite, anhydrous calcium hydrogen phosphate; glass fillers such as silica glass, aluminum-containing glass, barium-containing glass, strontium-containing glass and zirconium silicate glass; fillers for slowly releasing silver; fillers for slowly release fluorine. These inorganic fillers can be used alone or in combination.

In order to obtain a strong adhesion between the inorganic filler and the resin, it is preferable to use an inorganic filler which has undergone a surface treatment such as silane treatment or polymer coating.

In the composition of the present invention, the amount of filler (D) is preferably 20 to 150 parts by weight, more preferably 30 to 120 parts by weight, especially 35 to 100 parts by weight, and most preferably 40 to 80 parts by weight, based on 100 parts by weight of the total amount of the polymerizable monomer (A), the polymer powder (B) and the polymerization initiator (C).

### Other Components

The adhesive composition for hard tissue repair of the present invention may include, as desired, a polymerization inhibitor. As a specific example of the polymerization inhibitor, hydroquinone compounds such as hydroquinone and dibutylhydroquinone; phenols such as hydroquinone monomethyl ether, 2,6-di-tert-butylphenol, 2,6-di-tert-butyl p-cresol, catechol, pyrogallol, benzoquinone, 2-hydroxybenzoquinone, p-methoxyphenol, tert-butyl catechol, butylated hydroxyanisole, butylated hydroxytoluene and tert-butylhydroquinone. The polymerization inhibitor can be used alone or in combination of two or more, preferably, the mixture of hydroquinone monomethyl ether and 2,6-di-tert-butyl p-cresol. Generally, the polymerization inhibitor is added to the polymerizable monomer (A). The polymerization inhibitor is added in an amount of from 10 to 1000 ppm, more preferably from 20 to 500 ppm, more preferably from 25 to 200 ppm, based on the polymerizable monomer (A).

The composition for hard tissue repair of the present invention may contain an ultraviolet absorber such as benzotriazole or the like, if desired. The amount of the ultraviolet absorber is added in an amount of preferably 5 to 500 ppm, more preferably 10 to 200 ppm, based on the polymerizable monomer (A).

As other components, antiinfective agent, antibacterial agent, antiviral agent, hemostatic agent, platelet activator, bone formation factor, bone growth factor, synthetic peptide with hemostatic effect, and other pharmaceutical or therapeutic components can also be included.

As an example of other component, a colorant can also be included to visually clarify the distinction between the composition and the surrounding hard tissue.

### Kit for Hard Tissue Repair

The composition for hard tissue repair of the present invention is generally used by mixing the components immediately prior to use to prepare the composition for hard tissue repair. The polymerizable monomers (A), the polymer powder (B), the polymerization initiator (C), the filler (D), and all other components included if desired can be contained in three or more members, respectively, and stored in the form of a kit for hard tissue repair. For example, a mixture of the polymerizable monomer (A) and other components (such as a polymerization inhibitor, an ultraviolet absorber, etc.) comprised if desired is contained in member 1; the polymer powder (B), or a mixture of the polymer powder (B) and the filler (D) and other components (such as an anti-infective agent, an antibacterial agent, a colorant, etc.) comprised if desired are contained in member 2; a mixture of the polymerization initiator (C) and other components comprised if desired is contained in member 3, these are three members. When mixing these components, the order of mixing is not limited. In view of the more excellent stability of the resulting composition for hard tissue repair, it is preferred that the polymerizable monomer (A) and other components comprised if desired of member 1 are first mixed with the polymerization initiator (C) and other components comprised if desired of member 3, then mixed with the polymer powder (B), or the polymer powder (B) and the filler (D) and other components comprised if desired of member 2.

As member 1 for containing the polymerizable monomer (A) and other components comprised if desired, a material for preventing volatilization and scattering is preferred. Examples can include a sealable resin container having gas barrier property, a metal-plastic composite laminated film such as a laminated film of an aluminum foil and a plastic film) bag, and a glass ampoule. As specific examples of the materials of the member 2 for storing the polymer powder (B), or the polymer powder (B) and the filler (D) and other components comprised if desired, examples can include a resin container with good sealing performance that can prevent moisture absorption, a glass container, nonwoven fabrics made of breathable resin that can be sterilized based on ethylene oxide (EO), hydrogen peroxide, and other gases, and sterile paper. As the member 3 for containing the polymerization initiator (C) and other components comprised if desired, it is preferred that the material can prevent it from contacting the air and escaping. Specific example can include a sealable metal container having a gas barrier property, a metal-plastic composite laminate film (such as a laminate film of an aluminum foil and a plastic film) bag, a glass ampoule, and the like.

The members of the kit for hard tissue repair can also be the members having the function of mixing the components and preparing the adhesive composition for hard tissue repair, and directly filling and coating it on the affected parts of hard tissues such as bone and cartilage, soft tissues, and prosthesis for repair such as titanium, ceramic and stainless steel (for example, an adhesive pusher (extruder), a mixing container, an adhesive injector, and a cartridge). For example, the polymerizable monomer (A) of the present invention and other components comprised if desired are contained inside the storage container of the adhesive pusher, the polymerizable monomer (A) and the polymerization initiator (C), polymer powder (B), or polymer powder (B) and filler (D) are contacted and mixed when using it, and the adhesive composition is directly filled or coated on the affected part or the prosthesis for repair; alternatively, the inside of the storage container as the adhesive pusher is divided into two parts by a partition wall or a spacer, the polymerizable monomer (A) and the polymerization initiator (C) and other components comprised if desired of the present invention are respectively contained in the two separated positions, when using it, the polymerizable monomer (A) can be contacted and mixed with the polymerization initiator (C) by breaking or moving the partition wall or removing the spacer, and then is contacted and mixed with the polymer powder (B), or the polymer powder (B) and the filler (D), and the adhesive composition is directly filled or coated on the affected part or the prosthesis for repair; alternatively, the inside of the storage container as the adhesive pusher is divided into three parts by a partition wall or a spacer, the polymerizable monomer (A), the polymer powder (B) or the polymer powder (B) and the filler (D), the polymerization initiator (C) and all other components comprised if desired of the present invention are respectively contained in the separated three or more positions above, when using it, the polymerizable monomer (A) can be contacted and mixed with the polymerization initiator (C) and the polymer powder (B) or the polymer powder (B) and the filler (D) by destroying or moving the partition wall or removing the spacer, and then the adhesive composition is directly filled or coated on the affected part or the prosthesis for repair.

The kit for hard tissue repair of the present invention may further comprise a disinfectant such as an alcohol, a solution for hard tissue and auxiliary prosthesis pretreatment for the purpose of improving the sealing.

When the components are contained in the kit for hard tissue repair, it is preferable that the components are sterilized by electromagnetic wave such as ultraviolet ray, ethylene oxide and the like in the condition of no deterioration of the components (e.g., curing of monomers).

### EXAMPLES

The present invention is described in further detail below on the basis of the examples, but is not limited by these examples.

The abbreviations used hereinafter refer to the following compounds, respectively
MMA: methyl methacrylate
4-META: 4-methacryloyloxyethyl trimellitic anhydride
p-(MMA/BuMA): copolymer of methyl methacrylate and butyl methacrylate (particle size 68 μm, MMA content is about 25% by weight)

### Example 1

### (1) Preparation of the Initiator 1

182 g tri-n-butylboron was injected into the reactor under nitrogen atmosphere, and the temperature of the reactant was kept not higher than 80° C. 74 g anhydrous n-butanol was gradually dropped under stirring, the reactant was heated in circulating state under stirring. After 24 hours of circulating, the heating was stopped to obtain a mixture of butoxydibutylboron. The above mixture of butoxydibutylboron was distilled under reduced pressure under nitrogen atmosphere, a fraction under 92-94° C./8 mm Hg was collected. The purity was 97.6% by gas chromatography via relative area analysis, the target butoxydibutylboron 1 was obtained.

Under nitrogen atmosphere, 100 parts by weight of the above butoxydibutylboron 1 was placed in a mixing container and 3 parts by weight of anhydrous ethanol was slowly dropped while stirring to obtain the target initiator 1, and the target initiator 1 was stored in a nitrogen box before use.

### (2) Ignition Test

0.5 ml of the polymerization initiator composition was dropped onto a filter paper (whatman, No. 3) at 23° C.±2° C., the filter paper was stood to observe whether the filter paper emitted smoke, coked and ignited.

### (3) Polymerization Initiator Measuring Test:

The dropping state of the polymerization initiator composition was judged with naked eyes by dropping it from the syringe. Good measurability: drop by drop, no drawing/bubble mixing; Poor measurability: continuous dripping of the liquid droplets or drawing/bubble mixing, or non-dripping.

### (4) Polymerization Activity Test (Determination of Cure Time):

1) In a room at 25±2° C., 0.18 g of monomer (composition: MMA/4-META-95/5, weight ratio), 2 drops (about 0.015 g) of the polymerization initiator were dropped into a glass mixing dish, and 0.16 g of polymethacrylic acid (number average molecular weight: 400000, average particle size: about 25 μm) was further added, slightly mixed for 10 seconds to make resin mud;
2) A thin layer of vaseline was coated on a glass plate, a Teflon ring (outer diameter 13 mm, inner diameter 10 mm, thickness 5 mm) also coated with the thin layer of vaseline was placed on the glass plate, and the resin mud was poured on the glass plate;
3) within 30 seconds after the beginning of mixing, the glass plate containing the resin mud was transferred into a thermostat at 37±2° C. and 100% humidity. A probe was allowed to quietly fall on the surface of the test body to check whether the needle mark was formed. The time from the beginning of mixing to the time when no needle mark was formed on the test body was taken as the curing time.

The results were shown in table 1:

### Example 2

### Preparation of Initiator 2

182 g of tri-n-butylboron was injected into the reactor under nitrogen atmosphere; the temperature of the reactant was kept not higher than 40° C., the dry air was slowly blown on the surface of the reaction liquid while stirring; air corresponding to 0.5 molar equivalent of oxygen was blown in over about 6 hours to obtain the partially oxidized tributylboron. The above partially oxidized tributylboron was distilled under reduced pressure under nitrogen atmosphere, a fraction under 92-94° C./8 mm Hg was collected. The purity was 97.2% by gas chromatography via relative area analysis, the target butoxydibutylboron 2 was obtained.

Under nitrogen atmosphere, 100 parts by weight of the above butoxydibutylboron 2 was placed in a mixing container and 3 parts by weight of anhydrous ethanol was slowly dropped while stirring to obtain the target initiator 2, and the target initiator 2 was stored in a nitrogen box before use.

Except that, the evaluation was conducted the same as in example 1. The results were shown in table 1.

TABLE 1
Initiator for ignition safety, measurability, polymerization activity and other performances

| | Initiator/ | | | | | | Polymerization |
|---|---|---|---|---|---|---|---|
| | initiator | | | | | | activity |
| | composition | | | Safety against ignition | | | (curing |
| | (weight ratio) | Character | Measurability | Smoking | Coked | ignited | time) |
| Example 1 | Butoxydibutylboron 1/ | colorless | good | − | − | − | 8 min 30 s |
| | ethanol = 100/3 | liquid | | | | | |
| Example 2 | Butoxydibutylboron 2/ | colorless | good | − | − | − | 8 min 20 s |
| | ethanol = 100/3 | liquid | | | | | |
| Example 3 | Butoxydibutylboron 1/ | colorless | good | − | − | − | 8 min 50 s |
| | ethanol = 100/4 | liquid | | | | | |
| Example 4 | Butoxydibutylboron 1/ | colorless | good | − to + | − | − | 8 min 10 s |
| | ethanol = 100/0.5 | liquid | | | | | |
| Comparative | Mixture of | colorless | good | +++ | + | − | 8 min 0 s |
| example 1 | butoxydibutylboron | liquid | | | | | |
| Comparative | Partially oxidized | colorless | good | +++ | + | + | 7 min 40 s |
| example 2 | tributylboron | liquid | | | | | |
| Comparative | Mixture of | colorless | good | ++ | − | − | 8 min 20 s |
| example 3 | butoxydibutylboron/ | liquid | | | | | |
| | ethanol = 100/3 | | | | | | |
| Comparative | Partially oxidized | colorless | good | ++ | − | − | 8 min 0 s |
| example 4 | tributylboron/ | liquid | | | | | |
| | ethanol = 100/3 | | | | | | |
| Comparative | Butoxydibutylboron 1/ | colorless | good | − | − | − | about 12 min |
| example 5 | ethanol = 100/10 | liquid | | | | | |
| Comparative | Partially oxidized | viscous | bad | − | − | − | 9 min 0 s |
| example 6 | tributylboron/ | liquid | | | | | |
| | acetone/P-MMA/ | | | | | | |
| | BuMA = 100/56/30 | | | | | | |
| Comparative | Partially oxidized | colorless | good | − | − | − | 8 min 30 s |
| example 7 | tributylboron/ | liquid | | | | | |
| | n-hexane/ethanol = | | | | | | |
| | 100/20/3 | | | | | | |

In table 1, description of the symbols in the safety test sample for ignition:
Not smoking/not coked/not ignited: −
Coked/ignited: +
Amount of smoking: lot of +++; middle ++; small amount of +

### Examples 3 and 4

The evaluation was conducted the same as in example 1, except that butoxydibutylboron 1 and ethanol in the amounts shown in table 1 was used as the polymerization initiator (C). The results were shown in table 1.

### Comparative Example 1

The evaluation was conducted the same as in example 1, except that the mixture of butoxydibutylboron of example 1 above was used as the polymerization initiator (C). The results were shown in table 1.

### Comparative Example 2

The evaluation was conducted the same as in example 1, except that the partially oxidized tributylboron in above example 2 was used as the polymerization initiator (C). The results were shown in table 1.

### Comparative Examples 3-7

The evaluation was conducted the same as in example 1, except that the polymerization initiator having the composition and amounts shown in table 1 was used as the polymerization initiator (C). The results were shown in table 1.

### Example 5 (Adhesion Strength to Hard Tissue)

(1) Under the condition of water injection, the lip part of the ox anterior tooth was ground with coarse emery paper to expose the flat enamel surface, and then further polished with 600 # emery paper to form the adhesion surface. After dying the adhesion surface, it was treated with 35 wt. % phosphoric acid etching solution for 10 sec, washed with water for 10 sec, blow dried for 15 sec. Then, a hollow cellophane adhesive tape with a diameter of 4 mm was pasted on the adhesion surface to define the adhesion area.
(2) The resin mud was prepared at the same ratio as in the polymerization activity test of Example 1 (4).
(3) The resin mud was coated on the adhesion surface prepared by (1), and the acrylic rod was bonded to obtain a sample for adhesion test.

The sample for the adhesion test was placed at room temperature for 30 minutes, further impregnated in distilled water at 37° C. for 24 hours. The adhesion strength of the acrylic rod to the dental enamel was tested via a tensile test. The adhesion strength was the average of the values measured five times on the test samples. The adhesion strength was 10.9 MPa.

### Example 6 (Adhesion Strength to Hard Tissue)

The evaluation was conducted the same as in example 5, except that the polymerization initiator 2 in above example 2 was used as the polymerization initiator (C). The adhesion strength was 10.7 MPa.

Wherein, the composition for hard tissue repair of the present invention is generally used by mixing the components immediately prior to use to prepare the composition for hard tissue repair. The polymerizable monomers (A), the polymer powder (B), the polymerization initiator (C), the filler (D), and all other components included if desired can be contained in three or more members, respectively, and stored in the form of a kit for hard tissue repair. For example, a mixture of the polymerizable monomer (A) and other components (such as a polymerization inhibitor, an ultraviolet absorber, etc.) comprised if desired is contained in member 1; the polymer powder (B), or a mixture of the polymer powder (B) and the filler (D) and other components (such as an anti-infective agent, an antibacterial agent, a colorant, etc.) comprised if desired are contained in member 2; a mixture of the polymerization initiator (C) and other components comprised if desired is contained in member 3, these are three members. When mixing these components, the order of mixing is not limited. In view of the more excellent stability of the resulting composition for hard tissue repair, it is preferred that the polymerizable monomer (A) and other components comprised if desired of member 1 are first mixed with the polymerization initiator (C) and other components comprised if desired of member 3, then mixed with the polymer powder (B), or the polymer powder (B) and the filler (D) and other components comprised if desired of member 2.

As member 1 for containing the polymerizable monomer (A) and other components comprised if desired, a material for preventing volatilization and scattering is preferred. Examples can include a sealable resin container having gas barrier property, a metal-plastic composite laminated film (such as a laminated film of an aluminum foil and a plastic film) bag, and a glass ampoule. As specific examples of the materials of the member 2 for storing the polymer powder (B), or the polymer powder (B) and the filler (D) and other components comprised if desired, examples can include a resin container with good sealing performance that can prevent moisture absorption, a glass container, nonwoven fabrics made of breathable resin that can be sterilized based on ethylene oxide (EO), hydrogen peroxide, and other gases, and sterile paper. As the member 3 for containing the polymerization initiator (C) and other components comprised if desired, it is preferred that the material can prevent it from contacting the air and escaping. Specific example can include a sealable metal container having a gas barrier property, a metal-plastic composite laminate film (such as a laminate film of an aluminum foil and a plastic film) bag, a glass ampoule, and the like.

The members of the kit for hard tissue repair can also be the members having the function of mixing the components and preparing the adhesive composition for hard tissue repair, and directly filling and coating it on the affected parts of hard tissues such as bone and cartilage, soft tissues, and prosthesis for repair such as titanium, ceramic and stainless steel (for example, an adhesive pusher (extruder), a mixing container, an adhesive injector, and a cartridge). For example, the polymerizable monomer (A) of the present invention and other components comprised if desired are contained inside the storage container of the adhesive pusher, the polymerizable monomer (A) and the polymerization initiator (C), polymer powder (B), or polymer powder (B) and filler (D) are contacted and mixed when using it, and the adhesive composition is directly filled or coated on the affected part or the prosthesis for repair; alternatively, the inside of the storage container as the adhesive pusher is divided into two parts by a partition wall or a spacer, the polymerizable monomer (A) and the polymerization initiator (C) and other components comprised if desired of the present invention are respectively contained in the two separated positions, when using it, the polymerizable monomer (A) can be contacted and mixed with the polymerization initiator (C) by breaking or moving the partition wall or removing the spacer, and then is contacted and mixed with the polymer powder (B), or the polymer powder (B) and the filler (D), and the adhesive composition is directly filled or coated on the affected part or the prosthesis for repair; alternatively, the inside of the storage container as the adhesive pusher is divided into three parts by a partition wall or a spacer, the polymerizable monomer (A), the polymer powder (B) or the polymer powder (B) and the filler (D), the polymerization initiator (C) and all other components comprised if desired of the present invention are respectively contained in the separated three or more positions above, when using it, the polymerizable monomer (A) can be contacted and mixed with the polymerization initiator (C) and the polymer powder (B) or the polymer powder (B) and the filler (D) by destroying or moving the partition wall or removing the spacer, and then the adhesive composition is directly filled or coated on the affected part or the prosthesis for repair.

As described above, the present invention can provide a polymerization initiator which does not cause smoking, coking or exhibits ignition even when contacting paper, porous fibers or the like in the air, has high flowability, can be easily and accurately measured in a small amount, reduces adverse effects on the human body, and can impart high polymerization activity to the polymerizable composition, thereby an adhesive suitable for hard tissue repair is provided.

The above description is merely illustrative of the preferred embodiments of the invention and is not intended to limit the invention, it is intended that any modifications, equivalents, substitutions, and modifications made within the spirit and principles of the invention are embraced within the scope of the invention.

1. An adhesive composition for hard tissue repair, comprising:
a polymerizable monomer (A), a polymer powder (B), and a polymerization initiator (C), wherein the polymerization initiator (C) comprises 100 parts by weight of alkoxydialkylboron having a purity of 97% or more, 0.2-5 parts by weight of an alcohol having a boiling point of 60 to 180° C.;
wherein the polymerizable monomer (A) is a (meth)acrylate ester or a combination of a (meth)acrylate ester and a polymerizable monomer containing an acidic group; and
wherein the polymer powder (B) is at least one polymer selected from homopolymer of alkyl (meth)acrylate, copolymer between alkyl (meth)acrylates, copolymer of alkyl (meth)acrylate with other polymerizable monomer, copolymer of alkyl (meth)acrylate with alkylene di(meth)acrylate, and copolymer of alkyl (meth)acrylate with diene monomer.

2. The adhesive composition for hard tissue repair according to claim 1, wherein the alkoxydialkylboron comprised in the polymerization initiator (C) is butoxydibutylboron, the alcohol comprised in the polymerization initiator (C) is ethanol.

3. The adhesive composition for hard tissue repair according to claim 1, wherein the adhesive composition for hard tissue repair comprises 20-70 parts by weight of the polymerizable monomer (A), 20-70 parts by weight of the polymer powder (B), and 1-20 parts by weight of the polymerization initiator (C), wherein the total amount of the polymerizable monomer (A), the polymer powder (B), and the polymerization initiator (C) is 100 parts by weight.

4. The adhesive composition for hard tissue repair according to claim 3, wherein the adhesive composition for hard tissue repair further comprises 20-150 parts by weight of a filler (D) based on 100 parts by weight of the total amount of the polymerizable monomer (A), the polymer powder (B), and the polymerization initiator (C), wherein the filler (D) is an inorganic filler, an organic filler, or an organic-inorganic composite filler, which is insoluble or non-swellable in the polymerizable monomer (A).

5. A kit for hard tissue repair comprising the adhesive composition for hard tissue repair according to claim 1, wherein the polymerizable monomer (A) and part of the polymer powder (B) comprised in the composition for hard tissue repair are placed in the same member, the polymerization initiator (C) is placed in one member, the remaining part of the polymer powder (B) is placed in one member, and the three members above form the kit for repair; alternatively, the polymerizable monomer (A) and the filler (D) are placed in one member, the polymer powder (B) is placed in one member, the polymerization initiator (C) is placed in one member, and the three members above form the kit for repair; alternatively, the polymerizable monomer (A) is placed in one member, the polymerization initiator (C) is placed in one member, the polymer powder (B) and the filler (D) are placed in one member, and three members above forms the kit for repair.

6. A kit for hard tissue repair comprising the adhesive composition for hard tissue repair according to claim 2, wherein the polymerizable monomer (A) and part of the polymer powder (B) comprised in the composition for hard tissue repair are placed in the same member, the polymerization initiator (C) is placed in one member, the remaining part of the polymer powder (B) is placed in one member, and the three members above form the kit for repair; alternatively, the polymerizable monomer (A) and the filler (D) are placed in one member, the polymer powder (B) is placed in one member, the polymerization initiator (C) is placed in one member, and the three members above form the kit for repair; alternatively, the polymerizable monomer (A) is placed in one member, the polymerization initiator (C) is placed in one member, the polymer powder (B) and the filler (D) are placed in one member, and three members above forms the kit for repair.

7. A kit for hard tissue repair comprising the adhesive composition for hard tissue repair according to claim 3, wherein the polymerizable monomer (A) and part of the polymer powder (B) comprised in the composition for hard tissue repair are placed in the same member, the polymerization initiator (C) is placed in one member, the remaining part of the polymer powder (B) is placed in one member, and the three members above form the kit for repair; alternatively, the polymerizable monomer (A) and the filler (D) are placed in one member, the polymer powder (B) is placed in one member, the polymerization initiator (C) is placed in one member, and the three members above form the kit for repair; alternatively, the polymerizable monomer (A) is placed in one member, the polymerization initiator (C) is placed in one member, the polymer powder (B) and the filler (D) are placed in one member, and three members above forms the kit for repair.

8. A kit for hard tissue repair comprising the adhesive composition for hard tissue repair according to claim 4, wherein the polymerizable monomer (A) and part of the polymer powder (B) comprised in the composition for hard tissue repair are placed in the same member, the polymerization initiator (C) is placed in one member, the remaining part of the polymer powder (B) is placed in one member, and the three members above form the kit for repair; alternatively, the polymerizable monomer (A) and the filler (D) are placed in one member, the polymer powder (B) is placed in one member, the polymerization initiator (C) is placed in one member, and the three members above form the kit for repair; alternatively, the polymerizable monomer (A) is placed in one member, the polymerization initiator (C) is placed in one member, the polymer powder (B) and the filler (D) are placed in one member, and three members above forms the kit for repair.

9. The adhesive composition for hard tissue repair according to claim 2, wherein the adhesive composition for hard tissue repair comprises 20-70 parts by weight of the polymerizable monomer (A), 20-70 parts by weight of the polymer powder (B), and 1-20 parts by weight of the polymerization initiator (C), wherein the total amount of the polymerizable monomer (A), the polymer powder (B), and the polymerization initiator (C) is 100 parts by weight.

10. The adhesive composition for hard tissue repair according to claim 9, wherein the adhesive composition for hard tissue repair further comprises 20-150 parts by weight of a filler (D) based on 100 parts by weight of the total amount of the polymerizable monomer (A), the polymer powder (B), and the polymerization initiator (C), wherein the filler (D) is an inorganic filler, an organic filler, or an organic-inorganic composite filler, which is insoluble or non-swellable in the polymerizable monomer (A).

11. A kit for hard tissue repair comprising the adhesive composition for hard tissue repair according to claim 9, wherein the polymerizable monomer (A) and part of the polymer powder (B) comprised in the composition for hard tissue repair are placed in the same member, the polymerization initiator (C) is placed in one member, the remaining part of the polymer powder (B) is placed in one member, and the three members above form the kit for repair; alternatively, the polymerizable monomer (A) and the filler (D) are placed in one member, the polymer powder (B) is placed in one member, the polymerization initiator (C) is placed in one member, and the three members above form the kit for repair; alternatively, the polymerizable monomer (A) is placed in one member, the polymerization initiator (C) is placed in one member, the polymer powder (B) and the filler (D) are placed in one member, and three members above forms the kit for repair.

12. A kit for hard tissue repair comprising the adhesive composition for hard tissue repair according to claim 10, wherein the polymerizable monomer (A) and part of the polymer powder (B) comprised in the composition for hard tissue repair are placed in the same member, the polymerization initiator (C) is placed in one member, the remaining part of the polymer powder (B) is placed in one member, and the three members above form the kit for repair; alternatively, the polymerizable monomer (A) and the filler (D) are placed in one member, the polymer powder (B) is placed in one member, the polymerization initiator (C) is placed in one member, and the three members above form the kit for repair; alternatively, the polymerizable monomer (A) is placed in one member, the polymerization initiator (C) is placed in one member, the polymer powder (B) and the filler (D) are placed in one member, and three members above forms the kit for repair.
